(19) United States
(12) Patent Application Publication
(10) Pub. No.: US 2017/0340846 A1
(43) Pub. Date: Nov. 30, 2017

(54) INHALATION SYSTEM

(71) Applicant: PARI GmbH Spezialisten für effektive Inhalation, Starnberg (DE)

(72) Inventors: Jens Gramann, Gräfelfing (DE); Emir Jelovac, Munich (DE); Titus Selzer, Munich (DE); Nicolas Buchmann, Unterhaching (DE)

(73) Assignee: PARI GmbH Spezialisten für effektive Inhalation, Starnberg (DE)

(21) Appl. No.: 15/603,534

(22) Filed: May 24, 2017

(30) Foreign Application Priority Data
May 24, 2016 (EP) 16171017.3

(51) Int. Cl. A61M 16/00; A61M 16/14

(52) U.S. Cl. CPC A61M 16/0003; A61M 16/14; A61M 2205/3569; A61M 2205/3334; A61M 2205/3317; A61M 2205/0272; A61M 2016/003; A61M 2205/3592; A61M 2205/3327

(57) ABSTRACT

An inhalation system comprising an inhalation device and a flowmeter (12) that comprises a flow chamber (15), a resistor body (20) and a flow indicator (21), wherein said flow chamber (15) comprises an inlet opening (17) that can be connected to the surroundings, an outlet opening (18) that can be connected to an interior of the inhalation device, and a flow resistance device, said inhalation system being configured to guide supply air through the inlet opening (17) into the flow chamber (15), through the outlet opening (18) out of the flow chamber (15) and into the interior of the inhalation device, said resistor body (20) being configured to be able to assume different positions in the flow chamber (15), and said flow indicator (21) being configured to indicate a position of the resistor body (20) in the flow chamber (15).

The invention relates to an inhalation system comprising an inhalation device.

Inhalation systems are known in the prior art.

WO 2005/042075 A1 describes an inhalation therapy device comprising a nebulising chamber and an aerosol generator which is arranged such that it releases an aerosol into the nebulising chamber and which comprises a nozzle element. This inhalation therapy device does not, however, indicate whether the generated inspiratory flow is suitable for inhalation.

DE 197 34 022 C2 describes an inhalation therapy device comprising a valve for limiting the inspiratory flow. This inhalation therapy device indicates when the inspiratory flow is too great. It does not, however, indicate the targeted inspiratory flow and how the generated inspiratory flow relates thereto.

The object of the invention is to provide an inhalation system that indicates whether a generated inspiratory flow is suitable for inhalation.

The object is solved by an inhalation system comprising an inhalation device and a flowmeter that comprises a flow chamber, a resistor body and a flow indicator, said flow chamber comprising an inlet opening that can be effectively connected to the surroundings, an outlet opening that can be effectively connected to an interior of the inhalation device, and a flow resistance device, said inhalation system being configured to guide supply air through the inlet opening into the flow chamber, through the outlet opening out of the flow chamber and into the interior of the inhalation device, said resistor body being configured to be able to assume different positions in the flow chamber, and said flow indicator being configured to indicate a position of the resistor body in the flow chamber.

The inhalation device is preferably an aerosol generating device. An aerosol generating device preferably comprises a nebuliser, an atomiser, a humidifier, a compressed air nebuliser, an air atomiser, an electronic nebuliser, an ultrasonic nebuliser, an electrohydrodynamic nebuliser, an electrostatic nebuliser, a membrane nebuliser, a nebuliser having a vibrating membrane, an electronic nebuliser having a vibrating membrane, a mesh nebuliser, a nozzle nebuliser, an inhaler (MDI), a powder atomiser (DPI) or a combination thereof. In one embodiment, the inhaler comprises a pressurised canister comprising a medicament and a propellant. The canister is expediently connected to an actuator that can be operated by hand. It is advantageous for the inhaler to be configured so as to release a specific amount of medicament in aerosol form upon activation. In one embodiment, the aerosol generating device is configured for use with ventilators.

It is advantageous for the inhalation device to be a device for providing aerosols. The device for providing aerosols preferably comprises an inhalation aid, a spacer or a chamber. Devices for providing aerosols are preferably devices that are intended for use with inhalers (MDIs). They provide storage spaces for medicaments that are configured to accommodate aerosol preferably from inhalers such that it can be inhaled therefrom by users. Spacers do not comprise inhalation and exhalation valves, and a user should thus coordinate its breathing such that it does not exhale into the spacer. Chambers or holding chambers comprise exhalation and preferably also inhalation valves. It can thus be achieved that a flow of exhaled air is not guided into the space in which the aerosol is disposed. It can be achieved that a medicament can only exit the medicament storage space upon inhalation.

Aerosols are mixtures of solid or liquid suspended particles and a gas.

Aerosols are preferably provided for application on or in parts of the human or animal body such as the skin, body cavities, body orifices, the nose, the paranasal sinuses, the maxillary sinus, the frontal sinus, the sphenoidal sinus, the ethmoidal cells, the throat, the larynx, the trachea, the lungs, the stem bronchus, the bronchi, the bronchioles, the pulmonary alveoli, the joints or the abdominal cavity. Aerosols can be used to diagnose, prevent or treat diseases in humans and animals or to immunize humans or animals against diseases.

A flowmeter is a device that is configured to determine a property of a flow. The property is expediently a rate, a direction, a volume, a volume per unit of time or a mass. The flowmeter is preferably suitable for determining values, the exceeding of threshold values or the existence of specific ranges. In one embodiment, the flowmeter is configured to guide inspiratory air through the flow chamber in such a manner that it flows upwards therein. The flowmeter is preferably configured to divert the inspiratory air about a curve to the inhalation device. The flowmeter is expediently configured to supply inspiratory air to the inhalation device.

The flow chamber expediently comprises a hollow space. The hollow space preferably has a cone shape, a hollow cylindrical shape, a hollow conical shape or an arbitrary cross-section. It is expedient for the hollow space to have an elongated shape. In one embodiment, the hollow space has a continuously increasing cross-section. The cross-section expediently increases in a direction that is intended to be at the top, relative to the gravity of the earth, during operation. In one embodiment, the cross-section increases in an upwards slanting direction. The flow chamber preferably has a wall made of plastic, glass or metal. In one embodiment, the wall comprises a transparent region or is entirely transparent. It is particularly preferred for the wall to comprise a transparent plastic. It is particularly expedient for the wall to comprise an injection-moulded part. In one embodiment, the wall comprises a pipe. It is advantageous for the wall to have a rectangular or polygonal cross-section. In one embodiment, the flow chamber is integrated in another component of the inhalation system. The flow chamber is preferably integrated in a pipe of a nozzle nebuliser. The pipe is a component that is configured to guide ambient air to a nozzle. It is preferable for the ambient air to be guided to the nozzle by means of negative inspiratory pressure. A pressurised gas, preferably from a compressor or a gas cylinder, can expediently also be guided to the nozzle.

In one embodiment, the resistor body comprises a ball, a cone, a truncated cone, a cylinder or a combination thereof. The resistor body is preferably rotationally symmetric.

In one embodiment, the flow chamber and the resistor body are configured such that the resistor body can assume different positions in the flow chamber, influenced by the gravitational force and a gas that is intended to be able to flow through the flow chamber. The resistor body is expediently intended to be lifted with the inspiratory flow of a patient. The resistor body is preferably configured to float at different heights depending on the inspiratory flow.

A flow indicator is a device that is suitable for indicating a property of a flow. It is advantageous for the flow indicator to be configured to optically or acoustically indicate the property of the flow. In one embodiment, the flow indicator is configured to discretely indicate the property of the flow. It is preferred for the flow indicator to be configured to continuously indicate the property of the flow. In one embodiment, the flow indicator comprises a transparent region, through which the resistor body is visible at least in one position. It is expedient if the resistor body is visible in a position that indicates an optimal inspiratory flow. It is advantageous for the flow indicator to be configured to transmit the position of the resistor body to a mechanical or electrical display via a magnet system. By attaching the flow indicator in a region that is in the patient's field of vision in the operating state, the patient itself can receive feedback during inhalation as to the achieved inspiratory flow. The patient is the person who is using the inhalation system to guide a fluid out of the inhalation system onto or into parts of its body.

The flow resistance device preferably has a cross-section of the flow chamber that increases in the direction of opening. The cross-section preferably increases in the direction of flow. In one embodiment, the cross-section increases in the direction of the inhalation device. It is expedient for the cross-section to increase in the direction of the outlet opening. It is preferred for the cross-section to increase in a direction that is opposite to the earth's gravity when the inhalation system is in operation. In one embodiment, the cross-section increases in a direction that slants upwards when the inhalation system is in operation. In this manner, the resistor body can be moved against gravity by a flow.

In one embodiment, the flow resistance device comprises a spring or a magnet. The spring or magnet is expediently configured to be able to exert a force on the resistor body. It is preferred for the spring or magnet to be configured to exert a greater force on the resistor body as the flow through the flow chamber increases. It is expedient for the flow resistance device to be configured such that during operation, the air resistance on the resistor body works against the spring or the magnet. The resistor body is expediently configured to move further in the opening direction and to exert a greater force on the spring as the flow through the flow chamber increases. The movement of the resistor body can be appropriately coordinated by interpreting the spring characteristic curve. In one embodiment, the cross-section of the flow chamber is the same size in all areas that are intended to accommodate the resistor body.

The cross-section that increases in an opening direction is preferably realised by a cone-shaped configuration or by a cross-section that expands uniformly in all directions in the opening direction. The cross-section can be square, rectangular, triangular, oval or irregular. In one embodiment, the cross-section that increases in an opening direction is formed by the combination of a cross-sectional area that is constant in the opening direction and a cross-sectional area that increases in the opening direction. Both an edge of the constant cross-sectional area and an edge of the cross-sectional area that increases in the opening direction thereby preferably comprise a circular segment. The opening direction is expediently directed against the earth's gravity when the inhalation system is in operation.

Supply air preferably comprises ambient air. In one embodiment, supply air is intended to be supplied, in addition to another gas, to an interior of an inhalation device. The other gas is expediently a gas from an inhaler or a gas from a nozzle that is provided for aerosol generation. In one embodiment, the other gas also comprises ambient air and is intended to be guided directly out of the surroundings into the interior of the inhalation device. The supply air is expediently intended to be inhaled.

The inlet opening is preferably arranged such that it faces away from the user in the operating state. It can thus be prevented that exhaled air enters the measuring device and this device is contaminated, for instance, with residual aerosol. This is expediently realised by using an extension piece, preferably by using a hose.

An interior space of the inhalation device is preferably configured to accommodate or modify, such as mix, or convey fluids, such as liquids, gases or aerosols or a plurality thereof.

The inhalation system preferably comprises a plastic, particularly preferred polypropylene or polyamide. In one embodiment, the inhalation system comprises a transparent plastic. One or more components of the inhalation system preferably comprise plastic injection-moulded parts.

If the flow indicator is arranged within the field of vision or the range of audibility of a patient, the patient can observe and influence its inhalation behaviour in a particularly effective manner.

It is advantageous for specific flowmeters and nebuliser inserts to be geometrically coded such that specific flowmeters can only be used together with particular nebuliser inserts. In one embodiment, a specific flowmeter is connected to a particular nebuliser insert. Advantages can be achieved if the specific flowmeter is integrated in a nebuliser.

A nebuliser insert is an insert that can be introduced into a nebuliser. The nebuliser insert preferably comprises a filter that is configured to filter out droplets that exceed a predetermined value. The filter expediently comprises a baffle plate that is configured to guide the flow of aerosol such that droplets which exceed a predetermined size collide with the baffle plate and are removed from the aerosol.

It is advantageous for the flowmeter to be optimised for different flow ranges. In one embodiment, a light resistor body is provided, which is preferably intended for children. It is expedient for the gap between the resistor body and the flow chamber container to be adjusted to a specific flow range. This adjustment is preferably realised by specific diameters or inclines of the flow chamber container and specific diameters of the resistor body. It is expedient for the mass of the resistor body to be adjusted to the flow range.

In one embodiment, the inhalation system is configured to change the direction of flow of the supply air downstream of the flow chamber. In this manner, the flow indicator can be arranged by means of simple structural measures within the viewing range of a user during operation, such as in the field of vision of a patient. The flow indicator can be arranged such that it is located above the inhalation device during inhalation so that the patient can easily see the flow indicator during inhalation. The flow indicator can also be arranged in other favourable areas. The inhalation system can be configured to guide the flow around a curve once it has flown through the flow chamber or to guide the flow in a counter direction once it has flown through the flow chamber. A compact construction can thus be achieved.

In one embodiment, a pre-heating device is provided downstream of the flow chamber. The supply air can be preheated in this manner. By changing the direction of flow, the preheating device can be realised in a compact and effective manner.

By means of targeted changes in direction, it can be achieved that undesirable components in the supply air, such as dust or water droplets, do not reach the interior of the inhalation device.

It is expedient for the flow chamber to be configured such that the resistor body is enclosed in the flow chamber in an operating state of the inhalation system. It can thus be achieved that the resistor body remains in the flow chamber even if the inspiratory flow is too high or too low. The function of the flowmeter and of the flow indicator can thereby be maintained even if the inspiratory flow deviates greatly from an ideal inspiratory flow. The resistor body is enclosed if the inhalation system is configured to prevent the resistor body from leaving the flow chamber. The inhalation system is preferably configured to prevent the resistor body from leaving the flow chamber when a maximum inspiratory flow rate is exceeded or a minimum inspiratory flow rate is not achieved. The openings of the flow chamber are expediently so small that the resistor body cannot fit therethrough. In one embodiment, this is realised in that the inlet opening and the outlet opening have a cross-section that is smaller than the maximum cross-section of the resistor body. It is expedient if the flow chamber does not comprise any further openings other than the inlet opening and the outlet opening. In order to prevent the resistor body from leaving the flow chamber through the inlet opening or the outlet opening, a bar or projection can be provided. In one embodiment, the bar or projection is provided at the inlet or outlet opening or at both openings. The bar or projection can be provided in a region inside the flow chamber that is adjacent to the inlet or outlet opening or both openings.

In one embodiment, the resistor body is permanently enclosed in the flow chamber. In this manner, it is possible to prevent the resistor body from being accidentally swallowed. It is also possible to prevent the resistor body from being lost or accidentally swallowed during dismantling. The resistor body is permanently enclosed in the flow chamber if it cannot be removed from the flow chamber in a non-destructive manner. In one embodiment, this is realised in that one or more walls of the flow chamber form a cage or capsule. It is advantageous for a lid to be connected to a wall of the floating area with a form fit or a force fit (friction fit). One wall of the flow chamber expediently comprises a welding point, a soldering point, a glue point, a rivet or a catch. In one embodiment, a lid is welded to a wall of the flow chamber.

So that the resistor body cannot be accidentally swallowed or inhaled by playing children, a geometry can be inserted into the riser pipe, preferably following assembly of the resistor body, which prevents the resistor body from being able to be removed from the riser pipe. In one embodiment, this structure is integrated in a flexible member that can lock into place. In one embodiment, a lid is provided which is configured such that it cannot be removed, is preferably snap-locked, clip-locked or welded.

It is advantageous for the flowmeter to be configured such that it is removably connected to the inhalation device. This can facilitate cleaning. A removable connection is preferably realised by means of screws, a Velcro fastener, a friction fit, a form fit or a cone connection. In one embodiment, the flowmeter can be fitted to a pipe of a nozzle nebuliser via a cone. The inhalation device is preferably also able to function without the flowmeter. The flowmeter can thereby be used as an additional part that can be removed when it is not required. This could be the case if the patient has learned to generate a suitable inspiratory flow. The inhalation system is expediently configured such that the flowmeter can be removed and an inhalation valve can be attached. In one embodiment, the inhalation system is configured such that an inhalation valve can be attached in the region in which the flowmeter can be attached. It is advantageous for the inhalation system to be configured such that it at the same time comprises both a flowmeter and an inhalation valve that can be used independently thereof. It is particularly advantageous for the inhalation system to comprise an inhalation valve that is independent of the flowmeter. This means that the flowmeter can be removed in particularly simple manner and the inhalation system can be operated without the flowmeter. The flowmeter can be configured to be operated in bypass mode. In one embodiment, ambient air can be guided into the interior of the inhalation device both through the flowmeter and through an inhalation valve that is independent of the flowmeter. In a preferred embodiment, the flowmeter can be connected to the inhalation device such that the outlet openings of the flowmeter and the inhalation valve are connected in such a manner that a gas exiting the outlet opening can be guided to the inhalation valve. In a particularly expedient embodiment, the flowmeter can be connected to the inhalation system in such a manner that the inhalation valve is kept in a closed position if a flowmeter is connected to the inhalation system.

In one embodiment, the flow chamber comprises a guide device for the resistor body. Tilting of the resistor body can be better prevented in this manner. The remaining shape of the cross-section of the flow chamber can be designed comparatively independently of the guide function. A guide rib is preferably provided inside the flow chamber as the guide device. The guide rib is expediently arranged in the longitudinal direction. In one embodiment, a plurality of guide ribs, preferably three, are provided. In one configuration, the cross-section of the flow chamber comprises a first partial region with a guide device, which is largely constant in the opening direction, and a second partial region that widens in the opening direction. The guide device can comprise a part of a hollow cylinder.

It is advantageous for the resistor body and the flow chamber to be configured such that they reduce a flow cross-section in the flow chamber when a threshold flow is exceeded. As a result, the inhalation resistance becomes greater for the patient if the threshold flow is exceeded. In one embodiment, the resistor body and the flow chamber are configured to seal the flow cross-section in the flow chamber if the threshold flow is exceeded.

In order to be able to inhale a sufficient amount of inspiratory air through the inhalation system, the patient must inhale more slowly. In this manner, a user can be given specific feedback when the inspiratory flow is too high.

In one embodiment, the cross-section of the flow chamber in a region in which the resistor body is located upon reaching the threshold flow is smaller than in the region in which the resistor body is located in the case of an optimal inspiratory flow. The reduction in cross-section can occur in stages or continuously depending on the feedback desired for the patient. In one embodiment, the resistor body is configured to seal the outlet opening when the threshold flow is exceeded. A rubber seal can be provided at the outlet opening. The feedback for the patient can be influenced by the shape of the resistor body or of the rubber seal. A tapering form of the resistor body on the side facing the outlet opening can lead to an increase in the inhalation resistance up to a blocking of the inspiratory flow in that the outlet opening is closed further and further by the resistor body as the negative pressure increases. The tapering form preferably has a cone shape or a truncated cone shape. A flat shape of the resistor body on the side facing the outlet opening can lead to a sudden increase in the inhalation resistance with a quick blocking of the inspiratory flow or to a blocking of the inspiratory flow without a noteworthy prior increase in the inhalation resistance. The flat form is expediently a flat surface or a curved surface, whereby the curved surface has large radii of curvature.

In one embodiment, the flow chamber and the resistor body are configured to close the inlet opening of the flow chamber if a predetermined pressure difference between an interior of the flow chamber and the surroundings is not reached. It is advantageous for the flow chamber and the resistor body to be configured to close the inlet opening of the flow chamber when an overpressure occurs in the interior of the flow chamber. It can thereby be prevented during breathing pauses and upon exhalation that aerosol escapes from the inhalation system or that the compressor pushes aerosol into the flow chamber and contaminates it, changes the dimensions of the resistor body or cross-sections in the flow chamber, or that a transparent region of a wall of the flow chamber fogs up. It can also be prevented that a user exhales through the flow chamber.

The inlet opening of the flow chamber is closed when no or virtually no ambient air can reach the flow chamber through the inlet opening.

In one embodiment, the inlet opening is provided with a sealing surface or a seal, preferably with a rubber seal. It is advantageous for the resistor body to have a sealing surface or a seal. The inhalation system is expediently configured to be able to quickly close the inlet opening. In one embodiment, this is realised in that the resistor body comprises a flat or slightly curved surface in a region that can turn towards or is facing the inlet opening. In one embodiment, the resistor body has a ball shape or a cone shape. As a result hereof, the resistor body can close the inlet opening in a particularly simple and secure manner. The resistor body is expediently provided to fall downwards during exhalation or during breathing pauses, to block the inlet opening and to thus act as a valve. It is preferred for the resistor body to be configured to block the inlet opening during exhalation or during breathing pauses using the effect of gravity. In one embodiment, the resistor body is provided to close the inlet opening during exhalation or during breathing pauses using a spring force or a magnetic force and to thus act as a valve.

If the flow indicator is configured such that the resistor body, preferably the ball, does not completely close the inlet opening during exhalation, a PEP system can also be easily realised with the flow indicator. A PEP system has a positive expiratory pressure. This is generated by an increased exhalation resistance. It is particularly preferred for the flow indicator to be configured such that the function of a PEP system can be optionally activated or deactivated. A variable spacer is expediently provided for this purpose, which can be brought into a PEP position, preferably by a slider, in which it prevents the resistor body from completely closing the inlet opening.

It is expedient for the flow indicator to have a transparent region of a wall of the flow chamber. It can thereby be achieved in a simple manner that a user can see the position of the resistor body. It is expedient for the transparent region to be provided in a wall of the flow chamber. A region of the flow chamber is transparent if its light transmission is so great that a user can at least recognise whether the resistor body is in the transparent region. In one embodiment, a plurality of transparent regions are provided in the wall of the flow chamber. One of the transparent regions expediently indicates the optimal inspiratory flow. It is preferred for the light transmission to be so great that a user can recognise where the resistor body is located in the transparent region. In one embodiment, the flow chamber is completely transparent. It is advantageous for the flow chamber to comprise polypropylene, preferably transparent polypropylene or polyamide.

In one embodiment, the flow indicator is configured to transmit a position of the resistor body to a mechanical or electronic display via a magnet system. The magnet system expediently comprises an RFID system. In one embodiment, the resistor body is provided with a transponder. A reader is expediently provided on the flow chamber.

The flow indicator preferably comprises a marking. The marking is expediently provided on the transparent region. In one embodiment, the marking indicates a region in which the resistor body is located when there is an optimal inspiratory flow. It is advantageous for the marking to be adjustable or moveable. The marking is expediently configured as a clamp or clip. In one embodiment, the marking is provided as a label that is intended to be stuck on the inhalation system.

In one embodiment, the markings of specific flow indicators are coded according to the colours of corresponding nozzle inserts of the nebuliser, preferably the PARI LC Sprint. In one embodiment, the marking of a flow indicator intended for children is yellow like the yellow nebuliser insert of the PARI LC Sprint for children.

It is advantageous for the marking to comprise codeable symbols. A symbol can be assigned a meaning such as that there is an optimal inspiratory flow when the resistor body is located in the region of this symbol. The symbols can be assigned different meanings depending on the desired inspiratory flow. In one embodiment, the scale has the colours of a traffic light, i.e. green, yellow and red. It is advantageous for the scales to be exchangeable such that different scales can be used. The scales can preferably be clipped on with a clip or stuck on as a label. In this manner, different scales can be provided for different users such as for children or adults.

It is advantageous for the marking or scale to be configured to show where in the respiratory tract deposition is likely to take place. High flows are more likely to deposit in the upper respiratory tract, the bronchi and the larger bronchioles, whereas in the case of low flows, the periphery of the lungs and the alveoli can also be more easily reached. The marking or scale preferably uses numbers, colours or roughnesses, expediently produced using an erosion structure, for marking. In one embodiment, the markings or markers are raised or recessed, configured as a relief, printed or injection moulded in another colour. It is particularly preferred for the markings or markers to be injection moulded in a different colour to the surrounding area by means of a multi-component injection moulding process.

In one embodiment, the marking is configured as a scale. The scale expediently comprises lines. The scale is preferably configured such that it can be moved. If the inhalation device is a nozzle nebuliser and the scale specifies values for the volume per time of the flowing gas, it is preferably taken into consideration that gas from the aerosol generating nozzle is additionally supplied to the patient during inhalation. The aerosol generating nozzle is expediently supplied with a compressor flow of 2 to 6 l/m. The compressor flow is preferably 4 l/m. In this case, the patient is supplied with 4 l/m when the resistor body is in its lowermost position. It is therefore expedient for the scale to start at 4 l/m.

The marking is expediently configured as an orientation guide for a target inspiratory flow. The marking expediently comprises a target range. In one embodiment, the marking is intended to be attached to the inhalation system together with an expert. For logical reasons, the patient inhales with as ideal an inspiratory flow as possible before the marking is attached to the inhalation system. The expert can help the patient to achieve the ideal inspiratory flow. The marking can then be attached to the inhalation system such that the target range provided at the marking meets the ideal inspiratory flow.

The deliberate control of the respiratory flow is not a trivial matter. During the first few attempts, the ball can sometimes jump quite strongly. For many people, it is easier if the inhalation resistance is slightly higher and the diaphragm can work against this. In order to achieve this, a narrowing of the cross-section is preferably provided at a location of the airway. It is particularly preferred that the narrowing of the cross-section can be influenced such that different cross-sections can be set. In one embodiment, the narrowing of the cross-section is disposed directly at the inlet. The narrowing of the cross-section is preferably provided after the ball in the flow direction.

It is particularly expedient for the narrowing of the cross-section or restriction to be configured as a slider, a flap, an elastically deformable member or as a bypass opening similar to a flute hole. The latter is preferably positioned in front of the ball in the flow direction. An adjustment by means of a label on the flute hole, which can be removed after a training phase, is also advantageous. In one embodiment, a lid is used for reasons relating to demoulding in the region in which the flow direction is changed similar to in a siphon. In one embodiment, this lid is configured to be rotatable and restricts the air channel to a greater or lesser extent depending on its position.

It is advantageous for the restriction to have a rotatable member, by means of which a flow cross-section can be influenced. The rotatable member can expediently be at least partially rotated into and back out of the flow cross-section.

In one embodiment, the flowmeter is divided into two flow channels in a plane of the main axes. The two halves are preferably welded or clipped together. In one embodiment, the two halves are connected by a film hinge.

In one embodiment, the flowmeter comprises an outlet valve, and no outlet valve is provided on the inhalation device, in particular on a mouthpiece of the inhalation device.

It is advantageous if a measuring method, in particular a capacitive measuring method or a measuring method based on magnetic field change, is provided for detection of the resistor body, preferably the ball. The inhalation system comprises a measuring device therefor. The flowmeter is preferably configured to be filmed during inhalation such that the position of the resistor body can be determined. It is advantageous for the flowmeter to be configured to be filmed with a smartphone during inhalation such that the position of the resistor body can be determined. It is particularly preferred for the components to have a high contrast so that the image quality, in particular the definition, does not have to be high. The smartphone is preferably configured to calculate the position of the resistor body from the image data and to display this on the smartphone display for improved presentation and feedback. It is advantageous for the smartphone to be configured to store the data. The smartphone is advantageously configured to transmit data to a receiver. In one embodiment, the smartphone is configured to receive the data of a sensor, preferably a capacitive or magnetic sensor. It is expedient if provision is made for the sending or receiving of data to occur by means of wireless communication, preferably Bluetooth or Wi-Fi. In one embodiment, the smartphone is configured to conclude the location of deposition and the amount of deposited medicament. The measured data can thus be wirelessly transmitted to a receiver. The mobile device is configured to specify the location of deposition and/or the amount of deposited medicament based on the measured data.

In the following, the invention will be described in more detail by means of embodiment examples and with reference to the enclosed drawings.

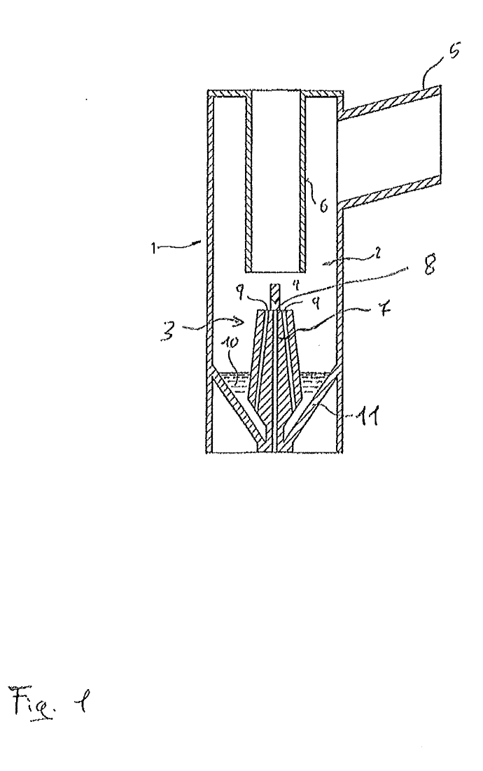

FIG. 1 shows an inhalation device from the prior art,

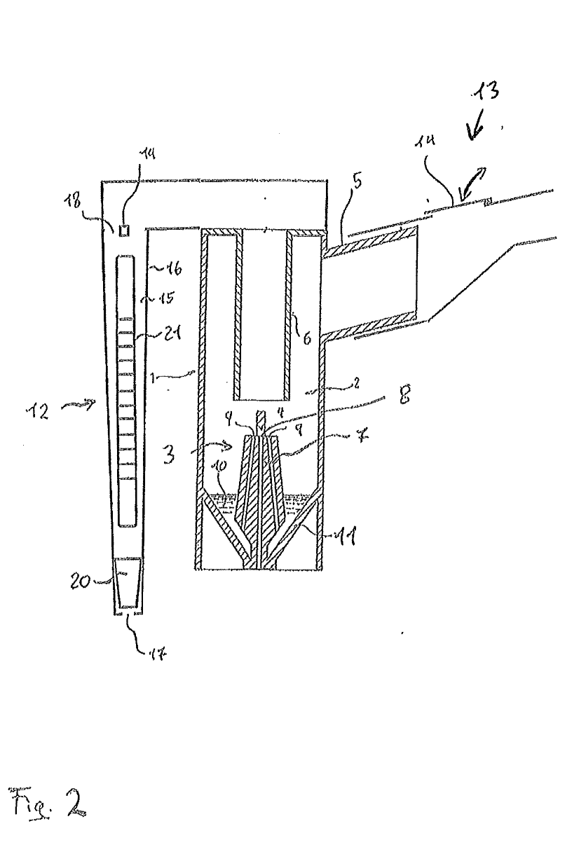

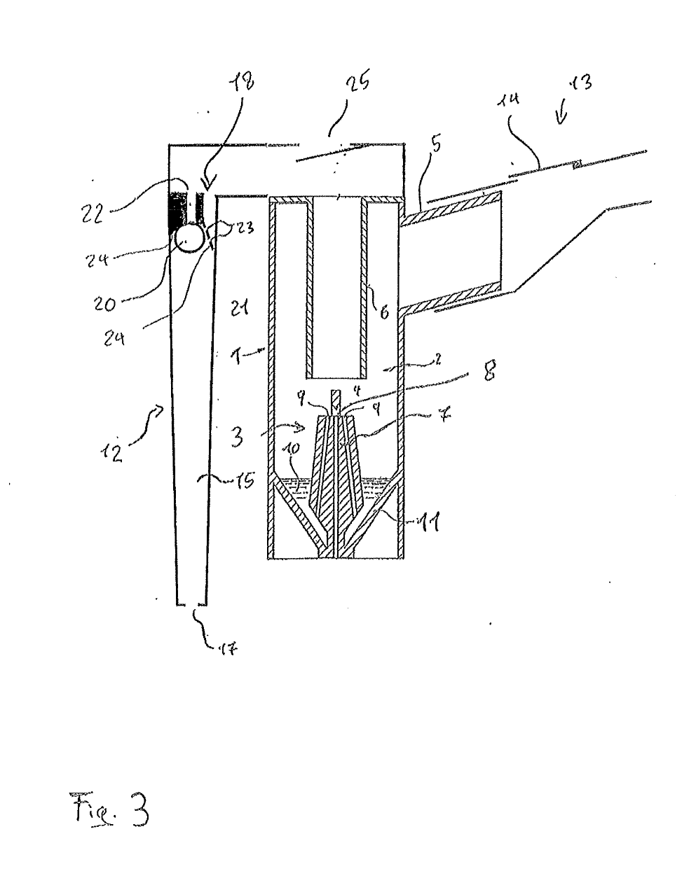

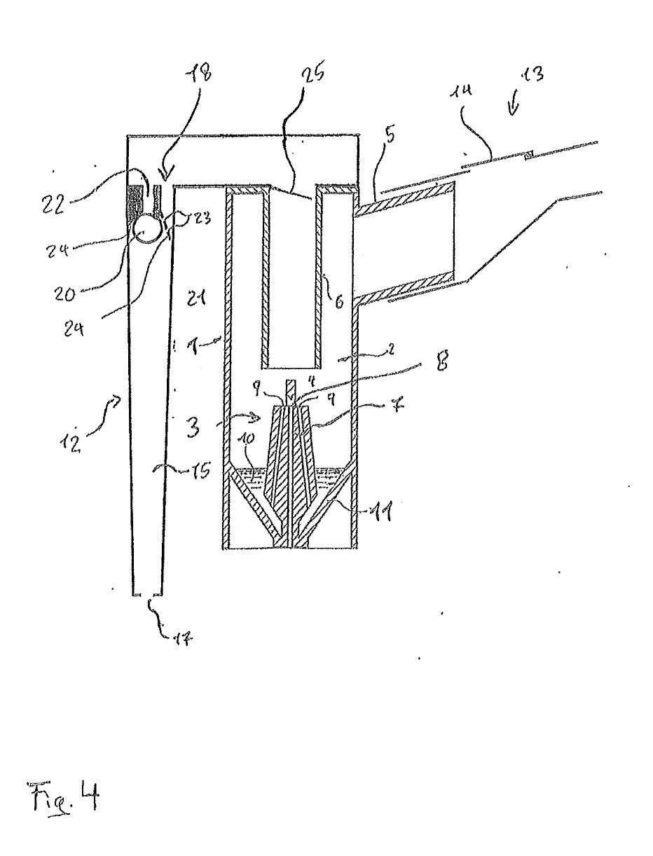

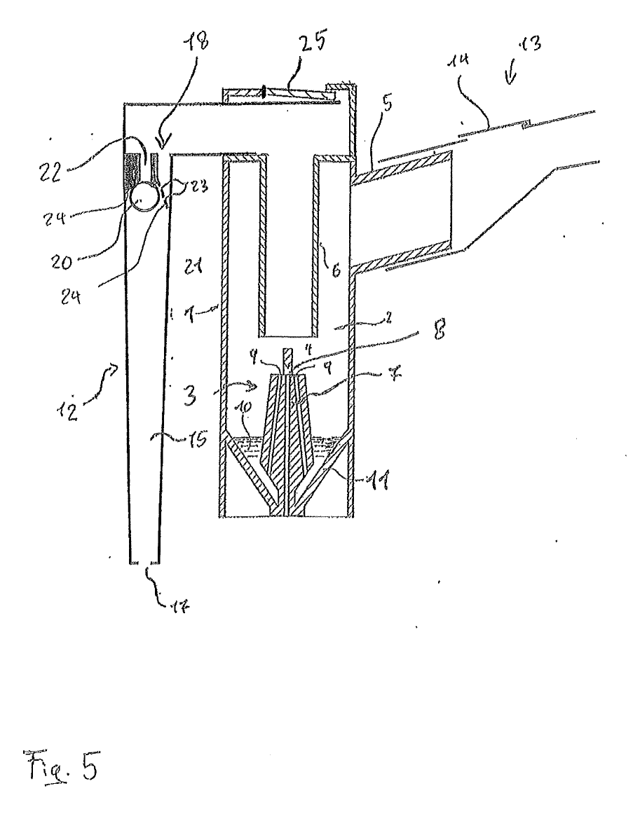

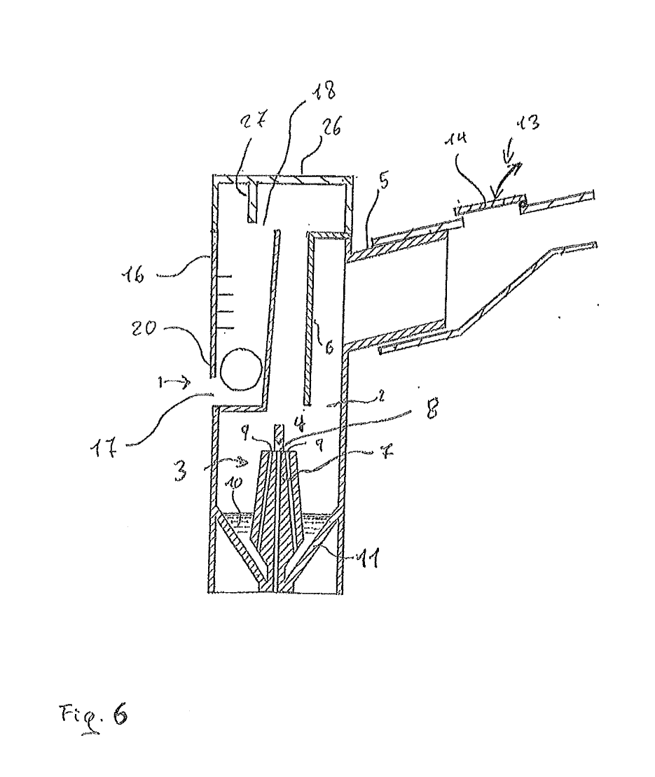

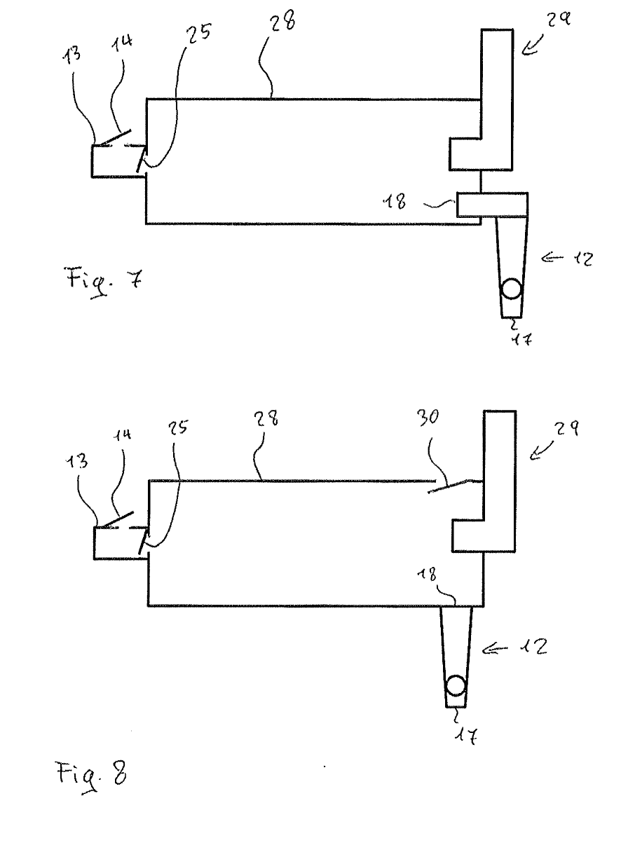

FIG. 2 shows an inhalation system comprising an inhalation device and a flowmeter,

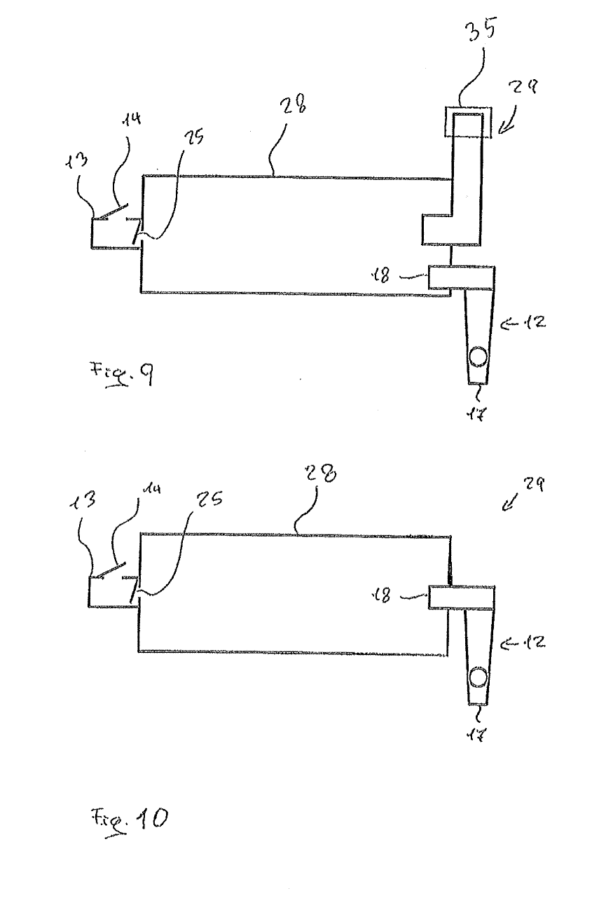

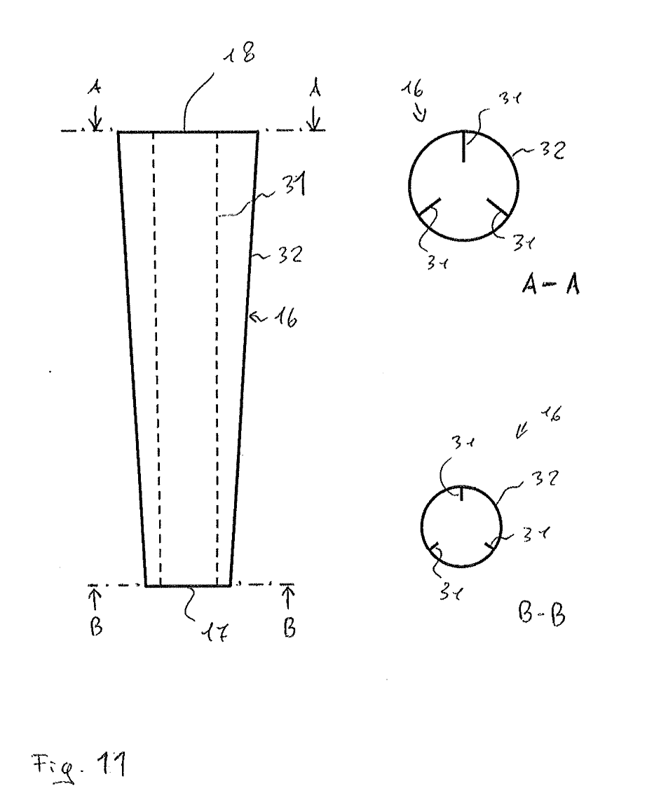

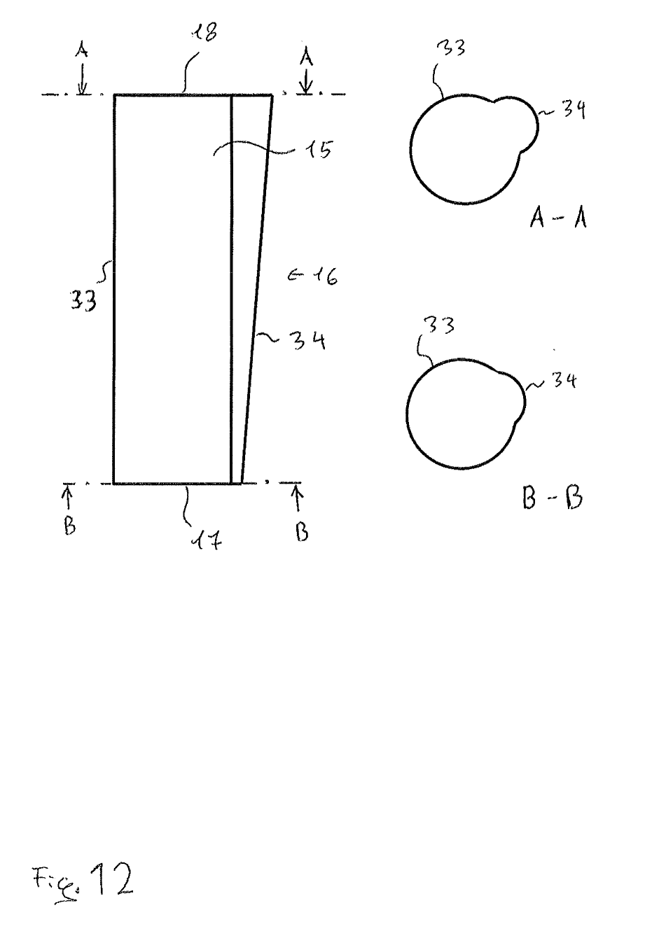

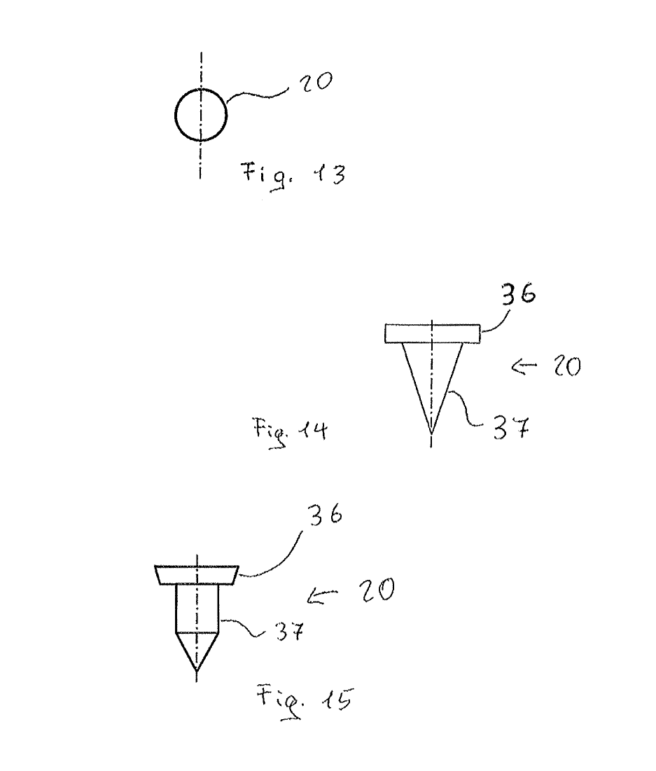

FIG. 3 shows an inhalation system comprising an inhalation device and a flowmeter, in which the resistor body and the flow chamber are configured to reduce a flow cross-section in the flow chamber when a threshold flow is exceeded,

FIG. 4 shows an inhalation system comprising an inhalation device and a flowmeter, in which the inhalation device can also be used without a flowmeter,

FIG. 5 shows a further inhalation system comprising an inhalation device and a flowmeter, in which the inhalation device can also be used without a flowmeter,

FIG. 6 shows an inhalation system, in which the flowmeter is integrated in the inhalation device,

FIG. 7 shows an inhalation aid comprising a flowmeter,

FIG. 8 shows an inhalation aid comprising a flowmeter, in which the flowmeter is disposed in bypass,

FIG. 9 shows an inhalation aid comprising a flowmeter, in which the inhaler is provided with a cap,

FIG. 10 shows an inhalation aid comprising a flowmeter, in which a flowmeter is positioned in a receiver that can also receive an inhaler,

FIG. 11 shows a flow chamber container comprising guide ribs,

FIG. 12 shows a flow chamber container comprising a guide contour,

FIGS. 13 to 15 show different resistor bodies,

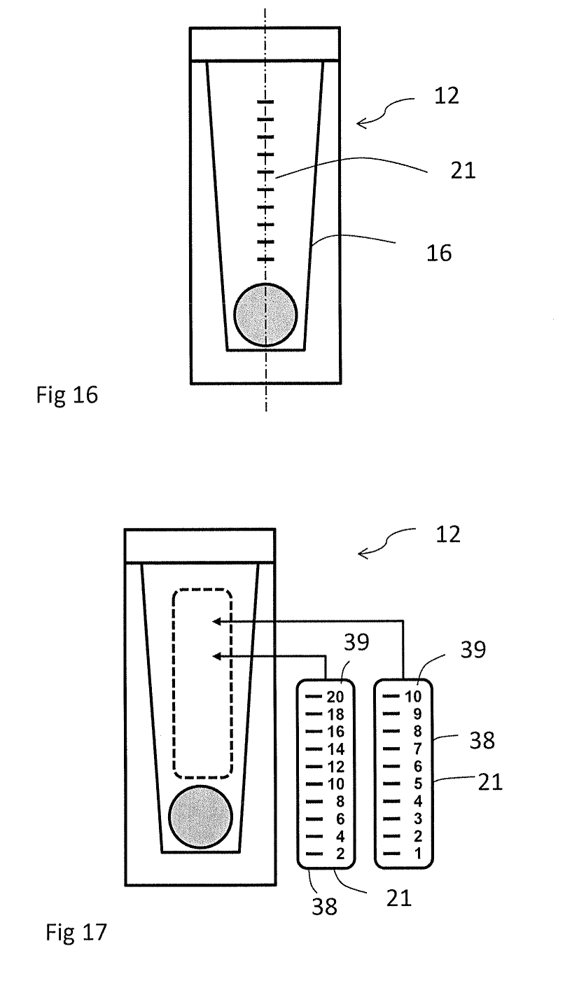

FIG. 16 shows a flowmeter with a scale,

FIG. 17 shows a flowmeter with different scales,

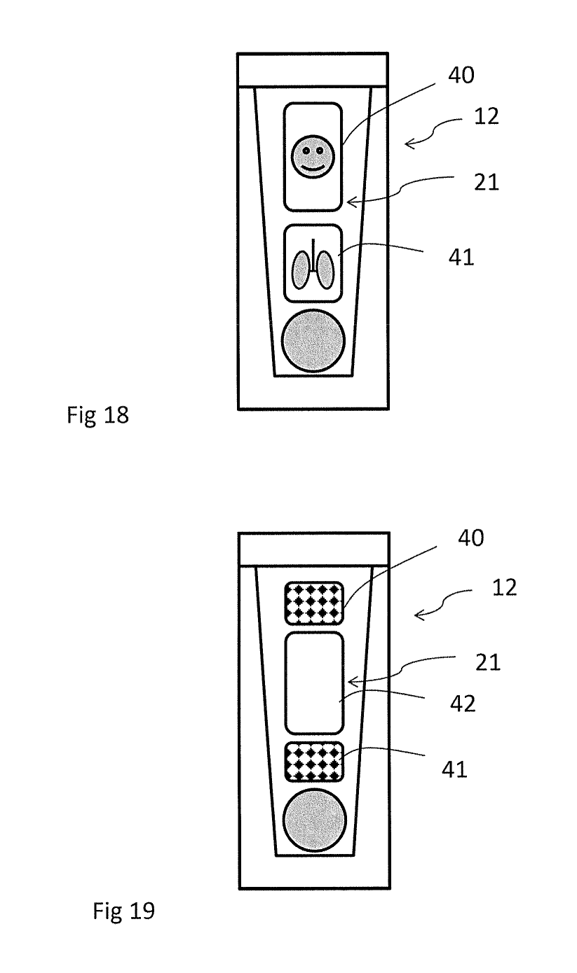

FIG. 18 shows a flowmeter with a specific scale,

FIG. 19 shows a flowmeter with a further specific scale,

FIG. 20 shows an inhalation system comprising coded flowmeters,

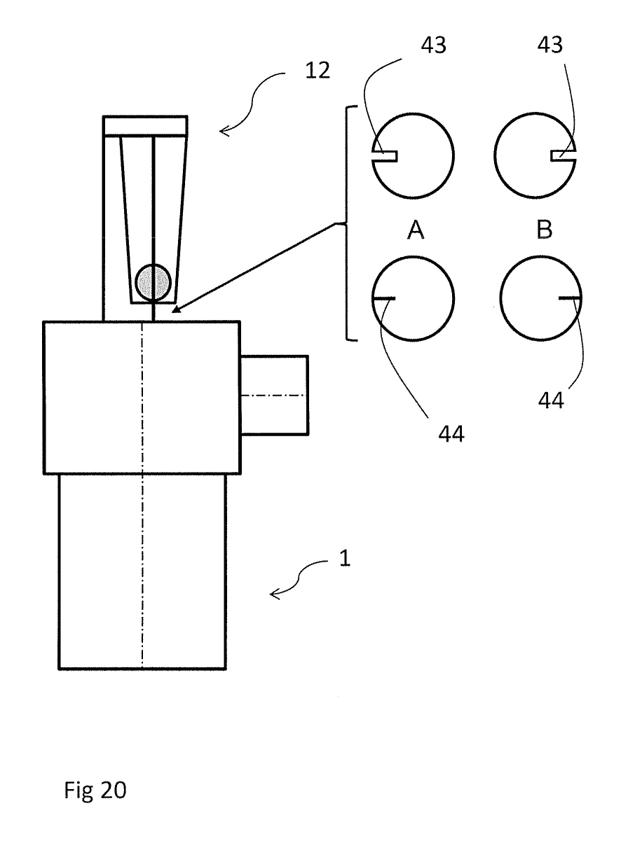

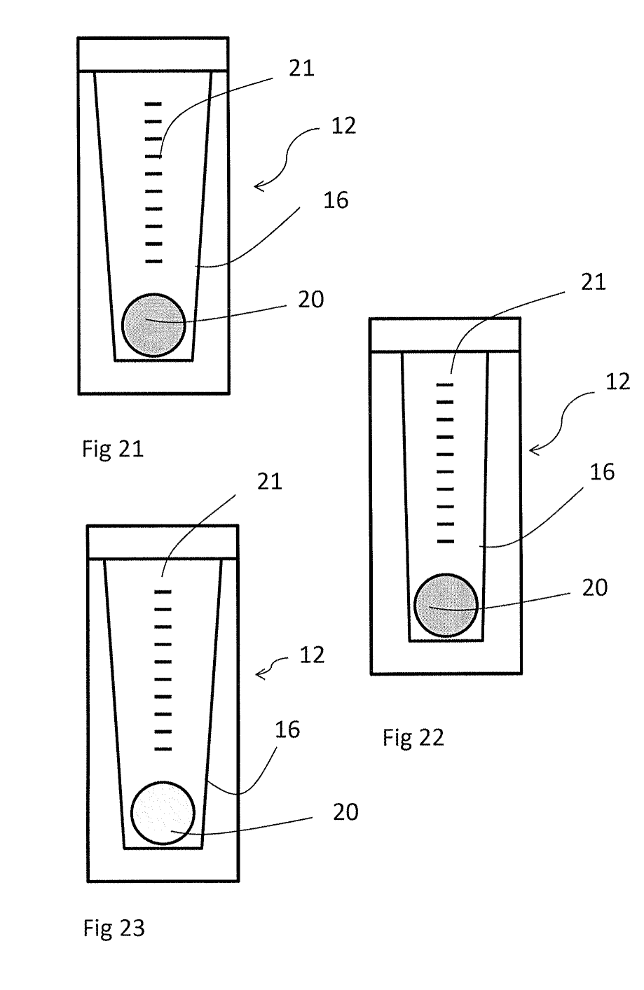

FIGS. 21 to 23 show flowmeters comprising different resistor bodies and flow chamber containers,

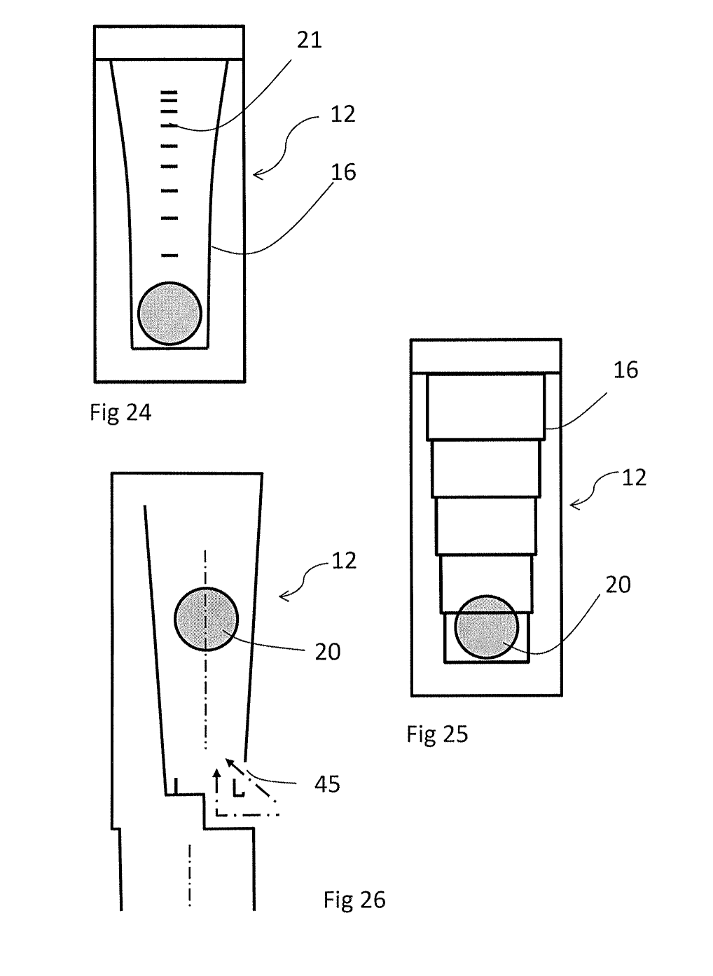

FIG. 24 shows a flowmeter comprising a flow chamber container having a non-linear cross-section,

FIG. 25 shows a flowmeter comprising a flow chamber container having a stepped cross-section,

FIG. 26 shows a flowmeter comprising a bypass opening,

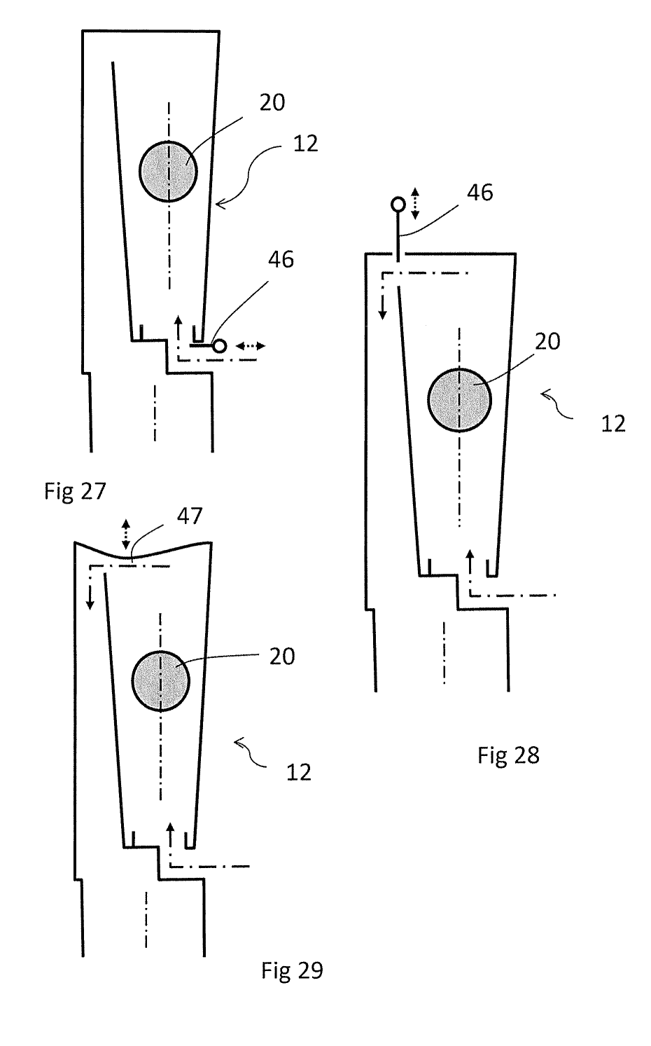

FIG. 27 shows a flowmeter comprising a restriction provided upstream of the resistor body,

FIG. 28 shows a flowmeter comprising a restriction provided downstream of the resistor body,

FIG. 29 shows a flowmeter comprising an elastically deformable member,

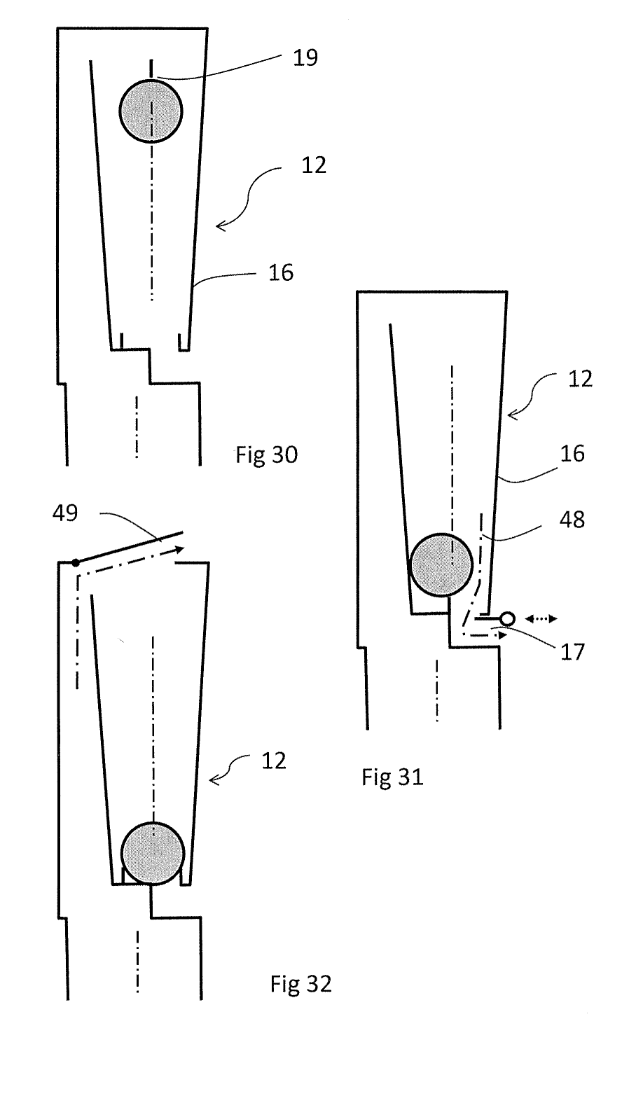

FIG. 30 shows a flowmeter comprising a bar in the flow chamber container downstream of the ball,

FIG. 31 shows a flowmeter comprising a device for positive expiratory pressure,

FIG. 32 shows a flowmeter comprising a separate exhalation valve, and

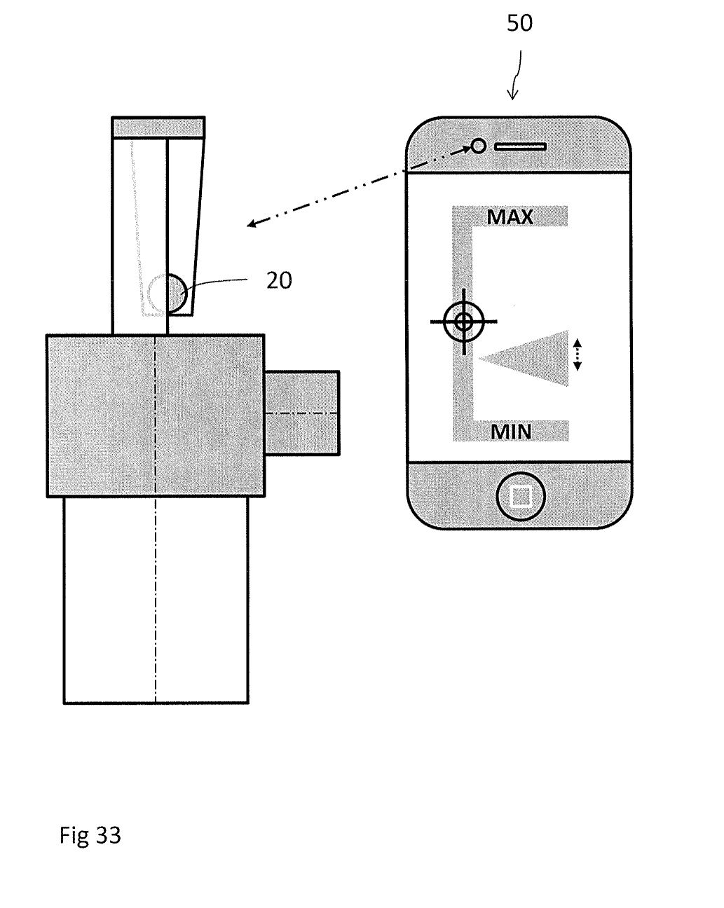

FIG. 33 shows an inhalation system, the resistor body of which can be detected with a smartphone.

FIG. 1 shows an inhalation device from the prior art. The inhalation device is a nozzle nebuliser 1 comprising a nebulising chamber 2. An aerosol generator 3, which is able to generate an aerosol 4, is provided in the nebulising chamber 2. An attachment 5 is provided on the nebulising chamber 2, to which, for example, a mouthpiece or mask that is not shown in the figure can be attached. The nebulising chamber 2 comprises a pipe 6, through which ambient air can be guided into the nebulising chamber 2.

The aerosol generator 3 comprises a nozzle element 7 with a nozzle opening 8, through which compressed air can be supplied in the present embodiment. The aerosol generator 3 furthermore comprises one or more channels 9, through which a medicament 10 can be guided out of a storage container 11 in the vicinity of the nozzle opening 8 out of which the compressed air guided through the nozzle element 7 escapes during operation. A mixture of compressed air and medicament is formed in this manner, which is released into the nebulising chamber 2. When a patient inhales through the attachment 5, for instance via a mouthpiece, ambient air is sucked into the nebulising chamber 2 through the pipe 6. This ambient air guides the generated aerosol 4 out of the nebulising chamber 2 to the attachment 5. The patient can inhale the aerosol 4 via a mouthpiece or mask connected to the attachment 5, which is not shown in the figure.

FIG. 2 shows an inhalation system comprising an inhalation device and a flowmeter 12. The inhalation device corresponds to the nozzle nebuliser 1 shown in FIG. 1. A mouthpiece 13 with an exhalation valve 14 is provided on the attachment 5 of the nozzle nebuliser 1. The double-headed arrow indicates the movement of the exhalation valve 14 during inhalation and exhalation.

The flowmeter 12 has a cone-shaped flow chamber 15, which is delimited by a flow chamber container 16 produced as an injected moulded part from polypropylene or polyamide. The flow chamber container 16 comprises an inlet opening 17 and an outlet opening 18. A bar 19 is provided in the outlet opening 18. A resistor body 20 is provided in the flow chamber 15, which also has a cone shape. A scale 21 is provided on the flow chamber container 16. The scale 21 is a printed label, on which lines and numbers (not shown in the figure) are provided, which indicate a flow through the nebulising chamber 2.

In FIG. 2, the resistor body 20 is shown in a position which it assumes during exhalation or a breathing pause of a patient. The resistor body 20 seals the flow chamber 15 at its perimeter such that no ambient air can reach the nebulising chamber 2 through the flowmeter 12. The air exhaled by the patient can be guided into the surroundings though the exhalation valve 14.

When the patient inhales through the mouthpiece 13, the exhalation valve 14 closes. Ambient air is guided as an inspiratory flow through the inlet opening 17 into the flow chamber 15.

Owing to the resulting pressure ratios, the resistor body 20 is moved with the airflow in the direction of the outlet opening 18 such that ambient air can flow between the resistor body 20 and the flow chamber container 16. The resistor body 20 assumes different positions in the flow chamber 15 depending on the strength of the generated inspiratory flow. The bar 19 thereby limits the area in which the resistor body 20 can move. Using the scale 21, it is possible to read how strong the generated inspiratory flow is by reading off the value at which the resistor body 20 is located. If the inspiratory flow is very high, the resistor body 20 will be prevented from leaving the flow chamber 15 by the bar 19.

The inspiratory flow flows through the outlet opening 18, out of the flow chamber 15 and into the pipe 6 of the nozzle nebuliser 1. The inspiratory flow is supplied here to the nozzle nebuliser 1 in the manner described in connection with FIG. 1.

The flowmeter 12 can, of course, also be used with other inhalation devices such as, for example, mesh nebulisers.

FIG. 3 shows an inhalation system comprising an inhalation device and a flowmeter 12, in which the resistor body 20 and the flow chamber 15 are configured to reduce a flow cross-section in the flow chamber 15 when a threshold flow is exceeded.

The inhalation system shown in FIG. 3 essentially corresponds to the inhalation system shown in FIG. 2. The differences will be explained below.

The resistor body 20 is configured as a ball in FIG. 3. The outlet opening 18 comprises a plurality of partial openings. One main opening 22 and two ancillary openings 23 are provided. A guide device 24 for the resistor body 20 is furthermore provided. The guide device 24 essentially has a funnel shape which extends up to the main opening 22 in such a manner that the resistor body 20 seals this opening when there are high inspiratory flows. In the embodiment shown here, the ancillary openings 23 are provided in a wall of the guide device 24. This wall can also be configured as a perforated plate or a sieve or can comprise only one ancillary opening.

When there are high inspiratory flows or negative inspiratory pressures, the resistor body 20 is pulled into the guide device 24 and pressed against the main opening 22 such that it is sealed. Inspiratory air can now only flow through the smaller ancillary openings 23. As a result hereof, the inhalation resistance greatly increases for the patient, but he is still able to inhale. The patient thus receives feedback that he is inhaling to quickly or that the generated inspiratory flow or the negative inspiratory pressure is too great for effective inhalation. The patient can generate a slower inspiratory flow such that the resistor body 20 moves away from the main opening 22 and the flow cross-section for the inspiratory air becomes larger again. The inhalation resistance reduces as a result thereof.

It is also possible to provide no ancillary openings 23 such that no ambient air can reach the nebulising chamber 2 from the flowmeter 12 if the inspiratory flow is too high.

A further difference to the inhalation system shown in FIG. 2 is that the flowmeter 12 is arranged in bypass here. An inhalation valve 25 is disposed adjacent to the pipe 6. Inspiratory air can be guided into the nebulising chamber 2 both through the inhalation valve 25 and through the flowmeter 12.

FIG. 4 shows an inhalation system comprising an inhalation device and a flowmeter 12, in which the inhalation device can also be used without the flowmeter 12.

The inhalation system shown in FIG. 4 substantially corresponds to the inhalation system shown in FIG. 3. The differences will be explained below.

The inhalation device comprises an inhalation valve 25. The flowmeter 12 is detachably attached to the inhalation device such that a gas flowing through the outlet valve 18 is guided to the inhalation valve 25. The detachable connection is a plug-in connection that is not shown in FIG. 4.

By disconnecting the plug-in connection and removing the flowmeter 12, the inhalation device can also be operated without the flowmeter 12.

FIG. 5 shows a further inhalation system comprising an inhalation device and a flowmeter 12, in which the inhalation device can also be used without the flowmeter 12. The inhalation device also comprises an inhalation valve 25. The flowmeter 12 is also detachably attached to the inhalation device. The flowmeter 12 is, however, attached to the inhalation device in such a manner that the inhalation valve 25 is blocked. When the flowmeter 12 is detached from the inhalation device, the inhalation valve 25 is released such that it can open. The detachable connection of the flowmeter 25 and the inhalation device is solved in the embodiment shown in FIG. 5 by a plug-in connection that is not shown in the figure.

The inhalation device shown in FIG. 5 can be used without the flowmeter 12. When the flowmeter 12 is connected to the inhalation device, a flow of gas can be guided through the outlet opening 18 into the pipe 6 without having to pass through an inhalation valve. A patient does not have to overcome the force of the inhalation valve 25 during inhalation.

FIG. 6 shows an inhalation system, in which the flowmeter 12 is integrated in the inhalation device.

The flow chamber container 16 is integrated in the main housing of the nozzle nebuliser 1 adjacent to the pipe 6. The inlet opening 17 is provided at the side of the flow chamber container 16. The pipe 6 and the flow chamber container 16 are covered by a lid 26 such that a flow of air can flow from the outlet opening 18 of the flow chamber container 16, through the lid 26 to the pipe 6.

A lid pin 27 can be provided on an inside of the lid 26 to prevent the resistor body 20 from leaving the flow chamber when there are high inspiratory flows. In an embodiment not shown in FIG. 6, the height of the lid 26 and the flow chamber container 16 are configured such that the resistor body 20 cannot escape from the flow chamber.

The main housing of the nozzle nebuliser comprising the pipe 6 and the flow chamber container 16 is configured in the embodiment shown in FIG. 6 as an injection moulded part made of polypropylene.

FIG. 7 shows an inhalation aid comprising a flowmeter 12. An inhaler 29 and a flowmeter 12 are provided on an aerosol chamber 28 at the inlet side. The inhaler 29 and the flowmeter 12 are thereby connected with a rear wall of the aerosol chamber 28 in this embodiment. An inhalation valve 25 is provided on the outlet side, which is effectively connected with a mouthpiece 13 comprising an exhalation valve 14.

The flowmeter 12 functions in the same manner as the flowmeters 12 that are described in connection with FIGS. 2 to 6. The inhaler 29 is triggered for inhalation such that it releases a medicament into the aerosol chamber 28. The patient inhales through the mouthpiece 13, the exhalation valve 14 closes, the inhalation valve 25 opens, and the patient sucks the gas located in the aerosol chamber 28 through the mouthpiece so that he can inhale it. At the same time, ambient air flows through the inlet opening 17 into the flowmeter 12 and through the outlet opening 18 out of the flowmeter 12 and into the aerosol chamber 28. As is described in connection with FIGS. 2 to 6, the quantity of gas flowing through the flowmeter 12 can be read off. In addition to the quantity of gas flowing through the flowmeter 12, the patient is additionally supplied with a quantity of gas flowing through the inhaler 29. Furthermore, the quantity of gas from the inhaler 29 itself is supplied to the patient.

FIG. 8 shows an inhalation aid comprising a flowmeter 12, in which the flowmeter 12 is arranged in bypass. One difference to the arrangement shown in FIG. 7 is that in this embodiment, ambient air can flow into the aerosol chamber 28 not only through the flowmeter 12 and the inhaler 29, but also through the inlet valve 30. A further difference is that the flowmeter 12 is not arranged on the rear wall but rather on a side wall of the aerosol chamber 28.

FIG. 9 shows an inhalation aid comprising a flowmeter 12, in which the inhaler 29 is provided with a cap 35. The cap 35 seals the inhaler 29 in an airtight manner at its inlet region. This prevents ambient air reaching the aerosol chamber 28 through the inhaler 29. More precise information regarding the supplied amount of ambient air can thereby be achieved in a simple manner. Inaccuracies or adjustments owing to different sized passages for ambient air in different inhalers 29 can be avoided in this manner.

FIG. 10 shows an inhalation aid comprising a flowmeter 12, in which a flowmeter 12 is positioned in a receiver that can also receive an inhaler 29. When using the inhalation aid, the inhaler 29 is first of all placed in the receiver and the medicament is introduced into the aerosol chamber 28. The inhaler 29 is then removed and the flowmeter 12 is placed in the receiver. Inaccuracies or adjustments owing to different sized passages for ambient air in different inhalers 29 can be avoided also in this manner.

FIG. 11 shows a flow chamber container 16 comprising guide ribs 31. The outer wall 32 of the flow chamber container 16 is configured in a circular manner, whereby the diameter of the circle increases continuously from the inlet opening 17 to the outlet opening 18. The flow chamber container 16 is provided with three guide ribs 31 that extend from the inlet opening 17 to the outlet opening 18.

In the embodiment shown in FIG. 11, the guide ribs 31 are configured such that the triangle formed therebetween is the same size over the entire length. As a result thereof, a resistor body 20 can be guided between the ribs equally well over the entire length of the ribs. It is possible to prevent tilting.

Owing to the use of guide ribs 31, the outer wall 32 of the flow chamber container 16 can be designed independent of guide functions for the resistor body 20. It can have different cross-sections. It is, however, expedient for it to have a cross-sectional area that becomes continuously larger at least in the region in which a flow of gas is supposed to be measured with the aid of the resistor body 20.

FIG. 12 shows a flow chamber container 16 comprising a guide contour 33. The guide contour 33 also extends from the inlet opening 17 to the outlet opening 18. In this embodiment, the guide contour 33 has the contour of an unclosed circle in cross-section. The size of the guide contour 33 remains the same over the length of the flow chamber 15. The guide contour 33 is connected to a flow cross-section 34. The flow cross-section 34 also has the contour of an unclosed circle in cross-section. The cross-sectional area of the flow cross-section increases continuously from the inlet opening 17 to the outlet opening 18.

In other embodiments that are not shown here, the cross sections of the guide contour 33 and of the flow cross-section have different shapes such as, for example, triangular or square, or they have irregular shapes. The shapes of the cross-sections can also differ from one another.

FIGS. 13 to 15 show different resistor bodies 20.

FIG. 13 shows a spherical resistor body 20. This resistor body 20 is particularly suitable for preventing tilting.

FIG. 14 shows a rotationally symmetric resistor body 20. The resistor body 20 comprises a cylindrical guide section 36 and a conical stabilising section 37. The cylindrical section is configured to be able to abut an inner side of a flow chamber container 16. The conical section can, in some embodiments of the flowmeter 12, stabilise the resistor body 20 in its position by preventing tilting of the resistor body 20 owing to its weight.

FIG. 15 also shows a rotationally symmetric resistor body 20. This resistor body 20 also comprises a guide section 36 and a stabilising section 37. The guide section 36 is configured in the shape of a cone section. The stabilising section 37 has a cylindrical shape that is connected to a cone. In this embodiment as well, the guide section 36 is configured to be able to abut an inner side of a flow chamber container 16. In this embodiment as well, the stabilising section 37 can, in some embodiments of the flowmeter 12, stabilise the resistor body 20 in its position by preventing tilting of the resistor body 20 owing to its weight.

FIG. 16 shows a flowmeter 12 comprising a scale 21. The flowmeter 12 is similar to the flowmeter 12 as described in connection with FIG. 2. The scale 21 comprises lines that indicate different flows. The lines are formed by applying colour to an outer side of the flow chamber container 16. In an embodiment that is not shown, the flow chamber container 16 has, in the region of the lines, a higher surface roughness, raised or recessed structures or comprises a different material than is the case in the regions adjacent to the lines. In an embodiment that is not shown, the scale has been injection-moulded in a different colour to the region adjacent to the scale using a multicomponent injection moulding process.

FIG. 17 shows a flowmeter 12 comprising different scales 21. The scales 21 are provided with scale numbering 39 and are configured as labels 38. The first label 38 is intended for adults and the second label 38 is intended for children. Depending on whether an adult or a child is using the flowmeter 12, the corresponding label 38 can be attached. The label 38 for children has a finer scale 21 with lower scale values than is the case for the label 38 for adults. The label 38 for adults has a larger measuring range than the label 38 for children.

In an embodiment that is not shown, the scales 21 are designed to be exchangeable. They comprise a clip by means of which they can be clipped onto the flowmeter 12.

FIG. 18 shows a flowmeter 12 comprising a specific scale 21, which shows where in the respiratory tract deposition is likely to take place. An upper scale region 40 shows a symbol that indicates a high flow, which is more likely to deposit in the upper respiratory tract, the bronchi and the larger bronchioles. Here the symbol is a smiley. A lower scale region 41 shows a symbol that indicates low flows, with which the periphery of the lungs is more likely to be reached. Here the symbol is the lungs.

FIG. 19 shows a flowmeter 12 comprising a further specific scale 21 having an upper scale region 40, a lower scale region 41, and a middle scale region 42. The upper and lower scale regions 40, 41 are configured as a structure or relief such that they comprise raised and recessed areas. The middle scale region 42 comprises a polished surface such that the resistor body 20 is particularly visible in this region.

FIG. 20 shows an inhalation system comprising a coded flowmeter 12 and a coded inhalation device 1. The flowmeter 12 comprises a groove 43. The inhalation device 1 comprises a rib 44. There are different types of flowmeters 12 and inhalation devices 1. However, not every flowmeter 12 fits every inhalation device 1. The grooves 43 and ribs 44 are respectively arranged such that a flowmeter 12 can only be expediently connected to a suitable inhalation device. In the embodiment shown here, all flowmeters 12 can be connected to all inhalation devices 1; however, a flowmeter 12 can only be connected to an unsuitable inhalation device 1 the wrong way round such that it cannot be read.

FIGS. 21 to 23 show flowmeters 12 comprising different resistor bodies 20 and flow chamber containers 16. FIG. 21 shows a flowmeter 12 for an adult, which comprises a heavy ball 20 as resistor body 20 and a conical flow chamber container 16. A scale 21 is provided on an outer wall of the flow chamber container 16. The lines of the scale 21 have been provided on the flow chamber container 16 by sanding the respective surface areas such that the lines have a higher surface roughness than their surroundings.

FIG. 22 shows a flowmeter 12 for a child, which comprises a heavy ball 20 as the resistor body 20 and a conical flow chamber container 16, whereby the cross-section of the flow chamber container 16 does not increase as greatly in the flow direction as is the case in the flowmeter 12 shown in FIG. 21. As a result hereof, as the flow rate increases, the ball 20 rises up more quickly in the flow chamber container 16 than is the case in the flowmeter 12 for an adult that is shown in FIG. 21. The flow rate can thus be read more precisely. However, this means that only a smaller range can be read. As the flow rate increases, the ball reaches the upper end of the flow chamber container 16 more quickly than is the case in the flowmeter 12 shown in FIG. 21. As compared to the scale 21 shown in FIG. 21, the scale 21 has a finer division and a smaller range of the measurable flow rate.

FIG. 23 shows a flowmeter 12 for a child, which comprises a light ball 20 and a conical flow chamber container 16. A scale 21 is provided on an outer wall of the flow chamber container 16, which has been adjusted to the light ball 20. By providing the light ball 20 as the resistor body 20, the ball 20 rises high in the flow chamber container 16 at lower flows than the ball 20 shown in FIG. 21.

In this embodiment, the colour of the scale 21 corresponds to the colour of a nebuliser insert or nozzle insert that is intended for use in connection with this scale 21. The scale 21 is yellow. This facilitates the correct combination of nebuliser insert or nozzle insert and flowmeter 12.

FIG. 24 shows a flowmeter 12 comprising a flow chamber container 16 having a non-linear cross-section. The cross-section changes exponentially. The shape of the flow chamber container 16 resembles a trumpet funnel. The resolution of the scale 21 provided here is distorted as a result thereof such that the region in which lower flows are indicated is more spread out than the region in which higher flows are indicated. The flow in the region of lower flows can thus be indicated more clearly, in particular for children, such that it can be read more easily.

FIG. 25 shows a flowmeter 12 comprising a flow chamber container 16 having a stepped cross-section. The flow chamber container 16 or riser pipe comprises a sequence of cylindrical pipes. As a consequence, the resistor body 20 provided here dwells at a transition until the flow is so large or so small that the next cylinder is rushed through until the resistor body 20 again dwells at the next transition. The movement of the resistor body 20 can be attenuated as a result of this measure.

FIG. 26 shows a flowmeter 12 comprising a bypass opening 45. The bypass opening 45 is positioned in front of a resistor body 20 in the flow direction. It is formed similar to a flute hole such that a user can seal the bypass opening 45 with a finger. This increases the inhalation resistance. The deliberate control of the respiratory flow is not a trivial matter. During the first few attempts, the resistor body 20 can sometimes jump quite strongly. For many users, it is easier if the inhalation resistance is slightly higher and the diaphragm can work against this. This is achieved by closing the bypass opening 45. Following a training phase, closing of the bypass opening 45 can be dispensed with such that the inhalation resistance is no longer increased. Since the closed bypass opening 45 results in restriction prior to the resistor body 20 in the flow direction, the drop in pressure changes the flow conditions such that measurement is slightly less accurate.

In an embodiment that is not shown, an adhesive label is provided, which can be applied to the bypass opening 45 and removed following the training phase.

FIG. 27 shows a flowmeter 12 comprising a restriction provided upstream of a resistor body 20. The restriction is made possible by providing a slider 46. By moving the slider 46 into the flow, the inhalation resistance can be increased such that the effects described in connection with FIG. 26 can be achieved. After a training phase, the slider 46 can be pulled out of the flow again.

FIG. 28 shows a flowmeter 12 comprising a restriction provided downstream of the resistor body 20. As in the embodiment shown in FIG. 27, the restriction is made possible by a slider 46. Since the slider 46 is arranged downstream of the resistor body 20, the flow conditions in the region of the resistor body 20 are not changed.

FIG. 29 shows a flowmeter 12 comprising an elastically deformable member 47, which is configured as a cap. By pressing on the elastically deformable member 47, the flow rate downstream of a resistor body 20 can be restricted. The effects described in connection with FIG. 26 can also be achieved by means thereof. When the elastically deformable member 47 is not pressed inwards, the flow rate is not restricted.

FIG. 30 shows a flowmeter 12 comprising a bar 19 in the flow chamber container 16 downstream of the resistor body 20. This prevents the resistor body 20 from being swallowed by user.

FIG. 31 shows a flowmeter 12 comprising a device for positive expiratory pressure. A spacer 48 is provided in the flow chamber container 16, which is arranged such that the resistor body 20 cannot completely close the inlet opening 17. It is consequently possible to exhale through the flowmeter 12. A positive expiratory pressure or PEP results owing to the fact that only a small opening is present and an increased exhalation resistance occurs as a result thereof.

FIG. 32 shows a flowmeter 12 having a separate exhalation valve 49. A further exhalation valve 49 in the inhalation system is therefore not necessary. The exhalation valves disposed in the mouthpieces in the prior art can be dispensed with.

FIG. 33 shows an inhalation system, the resistor body 20 of which can be detected with a smartphone 50. The resistor body 20 is visible from the outside such that it can be filmed during inhalation with a smartphone 50. The smartphone 50 is configured to calculate the position of the resistor body 20 from the image data and to store this data. The smartphone 50 is configured to draw conclusions with regard to the location of deposition and the amount of deposited medicament.

### REFERENCE NUMBERS

1 Nozzle nebuliser
2 Nebulising chamber
3 Aerosol generator
4 Aerosol
5 Attachment
6 Pipe
7 Nozzle element
8 Nozzle opening
9 Channel
10 Medicament
11 Storage container
12 Flowmeter
13 Mouthpiece
14 Exhalation valve
15 Flow chamber
16 Flow chamber container
17 Inlet opening
18 Outlet opening
19 Bar
20 Resistor body
21 Scale
22 Main opening
23 Ancillary opening
24 Guide device
25 Inhalation valve
26 Lid
27 Lid pin
28 Aerosol chamber
29 Inhaler
30 Inlet valve
31 Guide rib
32 Outer wall of the flow chamber container
33 Guide contour
34 Flow cross-section
35 Cap
36 Guide section
37 Stabilising section
38 Label
39 Scale numbering
40 Upper scale region
41 Lower scale region
42 Middle scale region
43 Groove
44 Rib
45 Bypass opening
46 Slider
47 Elastically deformable member
48 Spacer
49 Exhalation valve
50 Smartphone

1. An inhalation system comprising:
an inhalation device and
a flowmeter that comprises a flow chamber, a resistor body and a flow indicator,
wherein said flow chamber comprises an inlet opening that can be connected to the surroundings, an outlet opening that can be connected to an interior of the inhalation device, and a flow resistance device,
said inhalation system being configured to guide supply air through the inlet opening into the flow chamber, through the outlet opening out of the flow chamber and into the interior of the inhalation device,
said resistor body being configured to be able to assume different positions in the flow chamber,
and said flow indicator being configured to indicate a position of the resistor body in the flow chamber,
wherein said flowmeter is optimised for a specific flow range.

2. The inhalation system according to claim 1, wherein the diameter and/or the incline of the flow chamber and/or the diameter and/or the mass of the resistor body is adjusted.

3. An inhalation system comprising:
an inhalation device and
a flowmeter that comprises a flow chamber, a resistor body and a flow indicator,
wherein said flow chamber comprises an inlet opening that can be connected to the surroundings, an outlet opening that can be connected to an interior of the inhalation device, and a flow resistance device,
said inhalation system being configured to guide supply air through the inlet opening into the flow chamber, through the outlet opening out of the flow chamber and into the interior of the inhalation device,
said resistor body being configured to be able to assume different positions in the flow chamber,
and said flow indicator being configured to indicate a position of the resistor body in the flow chamber, and
wherein said flow chamber comprises a narrowing of the cross-section.

4. The inhalation system according to claim 3, wherein the narrowing of the cross-section is located at the inlet and/or after the resistance meter in the flow direction.

5. The inhalation system according to claim 3, wherein the narrowing of the cross-section comprises a slider, a flap, an elastically deformable member, a rotatable member and/or a bypass opening.

6. An inhalation system comprising:
an inhalation device and
a flowmeter that comprises a flow chamber, a resistor body and a flow indicator,
wherein said flow chamber comprises an inlet opening that can be connected to the surroundings, an outlet opening that can be connected to an interior of the inhalation device, and a flow resistance device,
said inhalation system being configured to guide supply air through the inlet opening into the flow chamber, through the outlet opening out of the flow chamber and into the interior of the inhalation device,
said resistor body being configured to be able to assume different positions in the flow chamber,
and said flow indicator being configured to indicate a position of the resistor body in the flow chamber, and
wherein the position of the resistor body can be measured by a measuring device of the inhalation system.

7. The inhalation system according to claim 6, wherein the measurement is based on a change of a magnetic field and/or of a capacity and/or on a film recording.

8. The inhalation system according to claim 6, wherein the inhalation device transmits measured data to a mobile device, in particular a smartphone.

9. The inhalation system according to claim 6, wherein the inhalation device wirelessly transmits the measured data to a receiver.

10. The inhalation system according to claim 1, wherein the inhalation system is configured to change the direction of flow of the supply air downstream of the flow chamber.

11. The inhalation system according to claim 1, wherein the flow chamber is configured such that the resistor body is enclosed in the flow chamber in an operating state of the inhalation system.

12. The inhalation system according to claim 1, wherein the resistor body is permanently enclosed in the flow chamber.

13. The inhalation system according to claim 1, wherein the flowmeter is configured such that it is removably connected to the inhalation device.

14. The inhalation system according to claim 1, wherein the flow chamber comprises a guide device for the resistor body.

15. The inhalation system according to claim 1, wherein the resistor body and the flow chamber are configured to reduce a flow cross-section in the flow chamber when a threshold flow is exceeded.

16. The inhalation system according to claim 1, wherein the resistor body and the flow chamber are configured to close the inlet opening of the flow chamber if a predetermined pressure difference between an interior of the flow chamber and the surroundings is not reached.

17. The inhalation system according to claim 1, wherein the flow indicator comprises a transparent region.

18. The inhalation system according to claim 1, wherein the flow indicator comprises a marking.
